(19) United States
(12) Patent Application Publication
(10) Pub. No.: US 2024/0168022 A1
(43) Pub. Date: May 23, 2024

(54) Compositions, kits, and methods for anti-microbial serology assays using anti-human immunoglobulin antibody

(71) Applicant: Siemens Healthcare Diagnostics Inc., Tarrytown, NY (US)

(72) Inventor: Tie Wei, Wilmington, DE (US)

(73) Assignee: Siemens Healthcare Diagnostics Inc., Tarrytown, NY (US)

(21) Appl. No.: 18/552,167

(22) PCT Filed: Apr. 21, 2021
(86) PCT No.: PCT/US2021/070435

§ 371 Date: Sep. 22, 2023

(51) Int. Cl. G01N 33/569; B01L 3/00; G01N 33/58; G01N 33/68

(52) U.S. Cl. CPC G01N 33/56983; B01L 3/502715; G01N 33/582; G01N 33/6854; G01N 2333/165; G01N 2469/20

(57) ABSTRACT

Reagents, kits, and microfluidics devices are disclosed for detecting the presence and/or concentration of antibodies directed to microorganisms in human biological samples. Also disclosed are methods of production and use of the reagents, kits, and microfluidics devices. Anti-human immunoglobulin antibodies are utilized as a reagent in a bridging immunoassay for detection of the microorganisms.

### CROSS REFERENCE TO RELATED APPLICATIONS/INCORPORATION BY REFERENCE STATEMENT

Not Applicable.

### STATEMENT REGARDING FEDERALLY SPONSORED RESEARCH OR DEVELOPMENT

Not Applicable.

### BACKGROUND

The field of medical diagnostics utilizes many different forms of assay technologies. When a patient is suspected of being infected with a microorganism (such as, but not limited to, a bacteria or virus), an assay may be performed on a biological sample from the patient to detect antibodies directed to the microorganism that are being produced by the patient's immune system.

When detection of anti-viral or anti-bacterial antigen antibodies (such as, but not limited to, IgG, IgM, and/or IgA) in patient serum and plasma is desired, bridging serology assays have been employed, in which an immobilized viral/bacterial antigen and a labeled viral/bacterial antigen are often used to formulate the assay reagents. In another example, latex particle agglutination assays utilize viral/bacterial antigen-coated latex particles as a single reagent that aggregate in the presence of anti-viral/bacterial antigen antibodies in patient samples.

However, due to the uncertainty associated with antibody excess (i.e., the hook effect), the need for early detection of viral/bacterial infection, and low anti-viral/bacterial antibody titer in some patient samples, there is a need to reduce the amount of sample required for testing while also enhancing the signal generated by the assay.

Thus, there is a need in the art for new and improved assays for antibodies against microbial antigens that overcome the disadvantages and defects of the prior art. It is to such new and improved reagents, kits, microfluidics devices, and methods for detecting antibodies to microbial antigens that the present disclosure is directed.

### BRIEF DESCRIPTION OF THE DRAWINGS

FIG. 1 schematically depicts a SARS-COV-2 Total (COV2T) assay (Siemens Healthineers, Tarrytown, NY).

FIG. 2 schematically depicts one non-limiting embodiment of a serology assay format constructed in accordance with the present disclosure.

FIG. 3 schematically depicts another non-limiting embodiment of a serology assay format constructed in accordance with the present disclosure.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, kits, devices, and/or methods disclosed herein can be made and executed without undue experimentation in light of the present disclosure. While the compositions, kits, devices, and/or methods have been described in terms of particular embodiments, it will be apparent to those of skill in the art that variations may be applied to the compositions, kits, devices, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halide selected from fluoride, chloride, bromide, or iodide, a C1-C4 compound selected one of the following: linear, branched or cyclic alkyl, optionally substituted, and linear branched or cyclic alkenyl, wherein the optional substituents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substituents are selected from one or more of alkenylalkyl, alkynylalkyl, cycloalkyl, cycloalkenylalkyl, arylalkyl, alkylaryl, heteroarylalkyl, heterocyclealkyl, optionally substituted heterocycloalkenylalkyl, arylcycloalkyl, and arylheterocycloalkyl, phenyl, cyano, hydroxyl, alkyl, aryl, cycloalkyl, cyano, alkoxy, alkylthio, amino, —NH (alkyl), —NH(cycloalkyl)₂, carboxy, and —C(O))-alkyl.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “antibody” is used herein in the broadest sense and refers to, for example, intact monoclonal antibodies and polyclonal antibodies, multi-specific antibodies (e.g., bispecific antibodies), as well as antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

The term “LOCI®” as used herein refers to a commercially used assay technique based on Luminescent Oxygen Channeling Assay (LOCI®) technology. The LOCI® advanced chemiluminescence assay is described, for example, in U.S. Pat. No. 5,340,716 (Ullman et al.), the entire contents of which are expressly incorporated herein by reference. The currently available LOCI® technology has high sensitivity and uses several reagents. In particular, the LOCI® assay requires that two of these reagents (referred to as a “sensibead” and a “chemibead”) be held by other specific binding partner assay reagents in a manner whereby the sensibead and chemibead are in close proximity to one another to achieve a signal. Upon exposure to light at a certain wavelength, the sensibead releases singlet oxygen, and if the two beads are in close proximity, the singlet oxygen is transferred to the chemibead; this causes a chemical reaction that results in the chemibead giving off light that can be measured at a different wavelength.

Turning now to certain non-limiting embodiments of the present disclosure, reagents, kits, and microfluidics devices are disclosed that can be utilized in serology assays for the detection of antibodies to microorganisms present in human biological samples. Anti-human immunoglobulin (Ig) antibodies are utilized as part of a second reagent in various serology assay formats in combination with a first reagent that contains at least one antigen to the microorganism. Anti-microbial antibodies present in the biological sample bind to the antigen of the first assay reagent, while the anti-human Ig antibodies present in the second reagent bind to the anti-microbial antibodies bound to the first assay reagent, thereby forming a detectable complex.

To detect anti-viral antigen antibodies (i.e., IgG, IgM, IgA, etc.) for the SARS-COV-2 virus in patient serum and plasma, a bridging immunoassay format is typically used that employs an immobilized viral antigen reagent and a labeled viral antigen reagent. Because one viral antigen needs to be immobilized on a bulky solid phase (such as, but not limited to, latex or magnetic particles), bridging two viral antigen molecules (with at least one attached to the solid phase) by a monomer bivalent antibody (such as IgG) may produce a weak signal due to slower Brownian movement of the solid phase antigen and the uncertainty of the anti-viral antibody orientation thereon. As a result, there is a need to enhance the detection signal of the bridging assay, especially for samples that have a low anti-viral antibody titer. In addition, it is possible that both binding arms of a patient's anti-viral antigen antibody could bind to multiple antigen molecules immobilized on the solid phase and thereby prevent detection of the antibody by the labeled antigen reagent.

Certain non-limiting embodiments of the present disclosure are directed to a kit for performing a serology assay for the detection of the presence and/or concentration of antibodies to a microorganism in a human biological sample. The kit includes at least two components: (a) a composition comprising at least one antigen of the microorganism directly or indirectly bound thereto; and (b) a composition comprising an anti-human immunoglobulin antibody directly or indirectly bound thereto, wherein at least one of (a) and (b) further comprises a detectable molecule. Anti-microorganism antibody present in the human biological sample binds to the antigen of (a), while the anti-human immunoglobulin antibody of (b) binds to the anti-microorganism antibody bound to (a), thereby forming a detectable complex.

Non-limiting examples of samples that may be utilized in accordance with the present disclosure (and thus upon which the formulation of a matrix may be based, in certain non-limiting embodiments) include biological samples such as, but not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.

The serology assay with which the reagents and kits of the present disclosure are utilized may detect human antibodies to an antigen of any microorganism for which detection is desired. For example (but not by way of limitation), the microorganism to be detected may be a bacterium, a virus, a protozoan, a fungus, or the like.

Non-limiting examples of bacteria that can be detected in accordance with the present disclosure include Acinetobacter, Actinomyces, Aeromonas, Aggregatibacter, Atopobium, Bacillus, Bacteroides, Bartonella, Bifidobacterium, Borellia, Brucella, Campylobacter, Chlamydia, Chlamydophila, Clostridium, Corynebacterium, Coxiella, Eikenella, Enterobacter, Enterococcus, Escherichia, Eubacterium, Francisella, Fusobacterium, Gardnerella, Haemophilis, Helicobacter, Klebsiella, Lactobacillus, Listeria, Mobiluncus, Moraxella, Mycobacterium, Mycoplasma, Neisseria, Parviomonas, Pasteurella, Porphyromonas, Prevotella, Propionibacterium, Proteus, Pseudomonas, Rickettsia, Salmonella, Serratia, Shigella, Staphylococcus, Streptococcus, Tannerella, Treponema, Vibrio, and Yersinia species, and the like.

Non-limiting examples of viruses that can be detected in accordance with the present disclosure include adenoviruses, astroviruses, coronaviruses (such as, but not limited to, severe acute respiratory syndrome coronavirus (SARS-COV) or Middle East respiratory syndrome coronavirus (MERS-COV)), Coxsackie viruses, cytomegaloviruses (CMV), echoviruses, encephalitis viruses, enteroviruses, Epstein-Barr viruses (EBV), erythroviruses, hantaviruses, hepatitis viruses, herpes viruses, human immunodeficiency viruses (HIV), influenza viruses, noroviruses, papilloma viruses, parainfluenza viruses, paramyxoviruses, polio viruses, rabies viruses, respiratory syncytial viruses (RSV), rhinoviruses, rotoviruses, rubella viruses, rubeola viruses, Varicella-Zoster viruses, West Nile viruses, and Zika viruses, and the like.

Non-limiting examples of protozoans that can be detected in accordance with the present disclosure include Ascaris, Babesia, Cryptosporidium, Cyclospora, Entamoeba, Enterobius, Giardia, Hymenolepis, Necator, Plasmodium, Strongyloides, Taenia, Toxoplasma, and Trichomonas species, and the like.

Non-limiting examples of fungi that can be detected in accordance with the present disclosure include yeasts, molds, and the like, including (but not limited to) Candida, Cryptococcus, Epidermophyton, Malassezia, Microsporum, and Trichophyton species, and the like.

In a particular (but non-limiting) embodiment, the microorganism detected by the serology assay is severe acute respiratory syndrome coronavirus (SARS-COV), severe acute respiratory syndrome coronavirus 2 (SARS-COV-2, the virus that causes COVID-19), Middle East respiratory syndrome coronavirus (MERS-COV), human coronavirus 229E (HCOV-229E), human coronavirus OC43 (HCoV-OC43), human coronavirus NL63 (HCoV-NL63), and human coronavirus HKU1 (HCoV-HKU1), HIV, Hepatitis B Core Total, Epstein-Barr virus, Herpes Virus (HSV), CMV, Rubella, H. pylori, or Toxoplasma gondii.

The antigen may be any antigen from the microorganism to be detected. Antigens that are useful in detection of each of the microorganisms listed above are well known in the art and widely available. In addition, the selection of antigens that may be utilized in accordance with the present disclosure is well within the purview of a person having ordinary skill in the art. Thus, no further disclosure thereof is deemed necessary.

In particular (but non-limiting) embodiments, the microorganism is SARS-COV-2, and the antigen is any SARS-COV-2 antigen known in the art or otherwise contemplated herein. For example (but not by way of limitation), the antigen may be from the nucleocapsid (N) protein, the spike (S) protein, the membrane (M) protein, the envelope (E) protein, the fusion (F) protein, and the like. In particular (but non-limiting) embodiments, the antigen may be from the nucleocapsid protein or the spike protein.

In one particular (but non-limiting) embodiment, the antigen is the receptor binding domain (RBD) of the S1 subunit of SARS-COV-2 spike protein. The RBD S1 antigen can be obtained from any source known in the art. For example (but not by way of limitation), this particular antigen is commercially available from GenScript (Piscataway, NJ); Meridian Life Sciences, Inc. (Memphis, TN); Sino Biological US Inc. (Wayne, PA); ACRO Biosystems (Newark, DE); Biorbyt, LLC (St. Louis, MO); Icosagen, AS (San Francisco, CA); and Bios Pacific Inc. (Emeryville, CA).

The anti-human Ig antibodies may specifically bind to any portion of any human immunoglobulin molecules known in the art or otherwise contemplated herein. For example (but not by way of limitation), the antibodies may be directed to human IgG, IgE, IgM, IgD, and/or IgA, and/or any portion thereof (such as, but not limited to, anti-human gamma chain, anti-human H+L, anti-human light chain, and the like). In a particular (but non-limiting) embodiment, the anti-human Ig antibody specifically binds to human IgA, human IgG, and human IgM; use of this antibody renders the assay a total anti-viral antigen antibody assay (i.e., a total SARS-COV-2 antibody assay).

Anti-human Ig antibodies (including, but not limited to, anti-human IgG, anti-human IgM, and/or anti-human IgA antibodies, as well as antibodies that recognize two or all three human immunoglobulin antibodies) are well known in the art, are widely commercially available, and have been vastly studied. For example (but not by way of limitation), a few commercial sources of anti-human IgG monoclonal and/or polyclonal antibodies include Rockland Immunochemicals, Inc. (Pottstown, PA); USBiological Life Sciences (Swampscott, MA); Santa Cruz Biotechnology, Inc. (Dallas, TX); Jackson Immuno Research Labs, Inc. (West Grove, PA); Thermo Fisher Scientific (Waltham, MA); and Sigma-Aldrich Corp. (St. Louis, MO). However, this list is not inclusive, and there are many additional commercial sources of anti-human Ig antibodies that can be utilized in accordance with the present disclosure. Thus, a person having ordinary skill in the art will clearly and unambiguously be able to identify and select a variety of anti-human Ig antibodies that can be utilized in accordance with the present disclosure, and as such, no further description of the anti-human Ig antibodies or the characteristics thereof is deemed necessary.

The composition (a) comprising the microorganism antigen and the composition (b) comprising the anti-human immunoglobulin antibody may possess any physical and/or structural characteristics that allow for detection of the binding thereto of antibodies to the microorganism. For example (but not by way of limitation), the composition may assume the form of a particle, a bead, a surface, or substrate, and the like. Compositions that may be utilized in serology assays are well known in the art and available commercially. Likewise, labels that may be utilized in such compositions are well known in the art and available commercially. Further, the selection of particular compositions and labels for a particular serology assay format is well within the purview of a person of ordinary skill in the art. As such, no further description thereof is deemed necessary. However, solely for the purposes of example, a few different serology assay formats and the particular compositions utilized therewith are provided herein below.

The kits of the present disclosure may be designed for use with any serology assay format known in the art or otherwise described herein.

The first composition utilized in the serology assay format is typically designed for isolation and/or detection of the complex formed by the bridging of the microbial antibody binding to the microbial antigen and the anti-human Ig antibody. In certain non-limiting embodiments, the first composition comprises an immobilized surface to which the at least one microbial antigen is directly or indirectly bound; as such, the first composition provides an immobilized surface on which the complex can be formed and thus isolated from the remainder of the sample and the non-bound components of the assay. However, the use of an immobilized surface as part of the first composition is for purposes of illustration only; the first composition can possess any physical and/or structural characteristics that allow for isolation and/or detection of the complex formed between an antimicrobial antibody and the first and second compositions. For example (but not by way of limitation), the first composition can also assume the form of a particle, a bead, an immobilized or non-immobilized surface or substrate, and the like.

In another particular (but non-limiting) embodiment of a serology assay format, the kits may further include one or more reagents for a chemiluminescent detection system, such as (but not limited to) a Luminescence Oxygen Channeling Assay (LOCI®) format. In this particular (but non-limiting) example, the kit includes a composition comprising a singlet oxygen-activatable chemiluminescent compound (such as (but not limited to) a chemibead) and a composition comprising a sensitizer (such as (but not limited to) a sensibead), wherein one of the reagents has a microorganism antigen directly or indirectly bound thereto, and the other reagent has an anti-human Ig antibody directly or indirectly bound thereto. In an alternative embodiment, the kit contains reagents for directly or indirectly attaching the composition(s) to the microorganism antigen and/or the anti-human Ig antibody, either prior to or during the assay.

In a particular (but non-limiting) embodiment, the first composition of the kit is further defined as comprising a singlet oxygen-activatable chemiluminescent compound having at least one antigen of the microorganism directly or indirectly bound thereto. The kit further contains a second composition that comprises a sensitizer capable of generating singlet oxygen in its excited state and at least one anti-human Ig antibody directly or indirectly bound to the sensitizer.

When the kit contains a second composition that comprises a sensitizer and the anti-human Ig antibody, the second composition may not be disposed in the kit with the anti-human Ig antibody already bound to the sensitizer. That is, rather than containing a single second composition that contains both sensitizer and anti-human Ig antibody, the kit may instead include two reagents: (i) a composition comprising a sensitizer capable of generating singlet oxygen in its excited state and having a biotin-specific binding partner directly or indirectly bound thereto; and (ii) at least one anti-human immunoglobulin antibody, wherein the antibody is biotinylated.

A chemiluminescent compound (chemiluminescer) is a compound that is chemically activatable and, as a result of such activation, emits light at a certain wavelength. Examples of chemiluminescers, by way of illustration and not limitation, include: olefins capable of reacting with singlet oxygen or a peroxide to form hydroperoxides or dioxetanes, which can decompose to ketones or carboxylic acid derivatives; stable dioxetanes which can decompose by the action of light; acetylenes which can react with singlet oxygen to form diketones; hydrazones or hydrazides that can form azo compounds or azo carbonyls such as (but not limited to) luminol; and aromatic compounds that can form endoperoxides, for example. As a consequence of the activation reaction, the chemiluminescers directly or indirectly cause the emission of light.

In certain embodiments, the singlet oxygen-activatable chemiluminescent compound may be a substance that undergoes a chemical reaction with singlet oxygen to form a metastabile intermediate species that can decompose with the simultaneous or subsequent emission of light. The composition comprising the chemiluminescent compound may be directly excited by the activated chemiluminescent compound; alternatively, the composition may further comprise at least one fluorescent molecule that is excited by the activated chemiluminescent compound.

A sensitizer is a molecule, usually a compound, that generates a reactive intermediate such as, for example, singlet oxygen, for activation of a chemiluminescent compound. In some non-limiting embodiments, the sensitizer is a photosensitizer. Other sensitizers that can be chemi-activated (by, e.g., enzymes and metal salts) include, by way of example and not limitation, other substances and compositions that can produce singlet oxygen with or without activation by an external light source. For example, certain compounds have been shown to catalyze the conversion of hydrogen peroxide to singlet oxygen and water. Non-limiting examples of other sensitizer substances and compositions include: oxides of the alkaline earth metals Ca, Sr, and Ba; derivatives of elements of groups 3A, 4A, SA, and 6A in d⁰ configuration; oxides of actinides and lanthanides; and oxidizers ClO⁻, BrO⁻, Au³⁺, IO₃⁻, and IO₄⁻; and in particular, molybdate, peroxomolybdate, tungstate, and peroxotungstate ions, and acetonitrile. The following references, which are hereby expressly incorporated by reference in their entirety, provide further disclosure regarding sensitizer substances and compositions that also fall within the scope of the present disclosure: Aubry, J. Am. Chem. Soc., 107:5844-5849 (1985); Aubry, J. Org. Chem., 54:726-728 (1989); Böhme and Brauer, Inorg. Chem., 31:3468-3471 (1992); Niu and Foote, Inorg. Chem., 31:3472-3476 (1992); Nardello et al., Inorg. Chem., 34:4950-4957 (1995); Aubry and Bouttemy, J. Am. Chem. Soc., 119:5286-5294 (1997); and Almeida et al., Anal. Chim. Acta, 482:99-104 (2003); the entire contents of each of which are hereby expressly incorporated herein by reference.

Also included within the scope of photosensitizers are compounds that are not true sensitizers but which on excitation by heat, light, ionizing radiation, or chemical activation will release a molecule of singlet oxygen. Members of this class of compounds include, for example (but not by way of limitation), the endoperoxides such as 1,4-biscarboxyethyl-1,4-naphthalene endoperoxide; 9,10-diphenylanthracene-9,10-endoperoxide; and 5,6,11,12-tetraphenyl naphthalene 5,12-endoperoxide. Heating or direct absorption of light by these compounds releases singlet oxygen.

A photosensitizer is a sensitizer for activation of a photoactive compound, for example, by generation of singlet oxygen by excitation with light. The photosensitizers are photoactivatable and include, e.g., dyes and aromatic compounds, and are usually compounds comprised of covalently bonded atoms, usually with multiple conjugated double or triple bonds. The compounds should absorb light in the wavelength range of from about 200 nm to about 1,100 nm, such as (but not limited to) a range of from about 300 nm to about 1,000 nm or a range of from about 450 nm to 950 nm, with an extinction coefficient at its absorbance maximum greater than 500 M⁻¹ cm⁻¹, or greater than 5,000 M⁻¹ cm⁻¹, or greater than 50,000 M⁻¹ cm⁻¹, at the excitation wavelength. Photosensitizers should be relatively photostable and may not react efficiently with singlet oxygen. Examples of photosensitizers, by way of illustration and not limitation, include: acetone; benzophenone; 9-thioxanthone; eosin; 9,10-dibromoanthracene; methylene blue; metallo-porphyrins such as (but not limited to) hematoporphyrin; phthalocyanines; chlorophylls; rose bengal; and buckminsterfullerene; as well as derivatives of these compounds.

Particular, non-limiting examples of chemiluminescent compounds and photosensitizers that may be utilized in accordance with the present disclosure are set forth in U.S. Pat. No. 5,340,716 (Ullman, et al.), the entire contents of which are hereby expressly incorporated herein by reference.

Any biotin-specific binding partners known in the art or otherwise contemplated herein may be utilized in accordance with the present disclosure. In certain non-limiting embodiments, the biotin-specific binding partner is an antibody against biotin. In other non-limiting embodiments, the biotin-specific binding partner is avidin or an analog thereof.

Any avidin analogs known in the art or otherwise contemplated herein may be utilized in accordance with the present disclosure, so long as the avidin or avidin analog is: (1) capable of association with the sensitizer; (2) capable of binding to the biotinylated analyte-specific binding partner; and (3) capable of binding to biotin that may be present in a sample. Non-limiting examples of avidin analogs that can be utilized in accordance with the present disclosure include those disclosed in Kang et al. (J Drug Target (1995) 3:159-65), the entire contents of which are expressly incorporated herein by reference. Particular non-limiting examples of avidin analogs include avidin, streptavidin, traptavidin, neutral avidin, Neutralite avidin, Neutravidin, Lite-avidin, succinylated avidin, other forms of modified or genetically engineered) avidin, esters, salts, and/or derivatives of any of the above, and the like.

Any fluorescent molecules known in the art that are capable of being excited by the activated chemiluminescent compound and emitting light at a particular, detectable wavelength can be utilized in accordance with the present disclosure as the fluorescent molecules of (a) and (b) (as well as (e), if present), so long as the signals produced by each fluorescent molecule is detectable from the signals produced by the other fluorescent molecules utilized. That is, the fluorescent molecule of (a) must emit light at a wavelength that is sufficiently different from the wavelength at which the fluorescent molecule of (b) emits light so that the two signals can be distinguished from one another when detected simultaneously. In a particular (but non-limiting) example, each fluorescent molecule utilized in accordance with the present disclosure is independently selected from the group consisting of terbium, uranium, samarium, europium, gadolinium, and dysprosium. For example (but not by way of limitation), terbium emits light at a wavelength of about 545 nm, uranium emits light at a wavelength of about 612 nm, and samarium emits light at a wavelength of about 645 nm.

It should be noted that the attachment of the antigen and anti-human immunoglobulin antibody to the other two components (i.e., the singlet oxygen-activatable chemiluminescent compound and sensitizer) described herein above is arbitrary; indeed, the present disclosure also includes the opposite of the attachments described herein above. That is, certain embodiments of the present disclosure include a kit that comprises: (a) a composition comprising a singlet oxygen-activatable chemiluminescent compound having at least one anti-human immunoglobulin antibody directly or indirectly bound thereto; and (b) a composition comprising a sensitizer capable of generating singlet oxygen in its excited state and an antigen of the microorganism directly or indirectly bound to the sensitizer. Alternatively, the kit may include (a) above in combination with (b) a composition comprising a sensitizer capable of generating singlet oxygen in its excited state and a biotin-specific binding partner directly or indirectly bound thereto, and (c) an antigen of the microorganism, wherein the antigen is biotinylated.

Therefore, certain non-limiting embodiments of the kits comprise: (a) a composition comprising a singlet oxygen-activatable chemiluminescent compound; (b) a composition comprising a sensitizer capable of generating singlet oxygen in its excited state; (c) at least one anti-human immunoglobulin antibody directly or indirectly bound to the chemiluminescent compound or the sensitizer; and (d) an antigen of the microorganism directly or indirectly bound to the chemiluminescent compound or the sensitizer to which (c) is not bound. Alternatively, the kit may include (a) a composition comprising a singlet oxygen-activatable chemiluminescent compound; (b) a composition comprising a sensitizer capable of generating singlet oxygen in its excited state and a biotin-specific binding partner directly or indirectly bound thereto; (c) at least one anti-human immunoglobulin antibody; and (d) an antigen of the microorganism; and wherein one of (c) and (d) is directly or indirectly bound to the chemiluminescent compound, while the other of (c) and (d) is biotinylated.

All of the kits of the present disclosure may be utilized for qualitative and/or quantitative measurements of anti-microbial antibodies.

The assay components/reagents present in the kits may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in microfluidics devices is described in detail in U.S. Pat. No. 9,244,085 (Samproni), the entire contents of which are hereby expressly incorporated herein by reference.

In addition to the assay components/reagents described in detail herein above, the kits may further contain other reagent(s) for conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Non-limiting examples of other reagents that can be included in the kits include wash solutions, calibration solutions, quality control solutions, dilution solutions, excipients, interference solutions, positive controls, negative controls, and the like. In addition, the kit can further include a set of written instructions explaining how to use the kit. A kit of this nature can be used in any of the methods described or otherwise contemplated herein.

Certain non-limiting embodiments of the present disclosure are directed to a microfluidics device that contains any of the serology assay reagents described or otherwise contemplated herein. For example (but not by way of limitation), certain additional non-limiting embodiments of the present disclosure are directed to a microfluidics device that includes the components of any of the kits described herein above.

In particular, certain non-limiting embodiments include a microfluidics device for detecting the presence and/or concentration of antibodies to a microorganism in a biological sample via the serology assays described or otherwise contemplated herein. The microfluidics device comprises (i) an inlet channel through which the biological sample is applied; and (ii) at least a first compartment capable of being in fluidic communication with the inlet channel. The compartment(s) of (ii) contains any of the reagents described or otherwise contemplated herein, either alone or in combination with one or more other reagents described or otherwise contemplated herein (such as, but not limited to, one or more reagents for use in the serology assay). For example (but not by way of limitation), the one or more reagents include the first and/or second compositions (containing the microbial antigen and the anti-human Ig antibody, respectively) as described herein above or otherwise contemplated herein.

In certain non-limiting embodiments, all of the reagents (as well as any additional elements, as described herein above) of (ii) are present in the same compartment. In alternative non-limiting embodiments, the reagents are split between two or more compartments.

The microfluidics device may be provided with any arrangement of compartments and distribution of the various components therebetween that allows the device to function in accordance with the present disclosure.

Any of the compartments of the microfluidics device may be sealed to maintain reagent(s) disposed therein in a substantially air tight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that each of the compartment(s) may still be sealed, but that the two compartments are capable of having fluid flow therebetween upon puncture of a seal formed therein or therebetween.

The microfluidics devices of the present disclosure may be provided with any other desired features known in the art or otherwise contemplated herein. For example, but not by way of limitation, the microfluidics devices of the present disclosure may further include a read chamber; the read chamber may be any of the compartments containing the reagents described herein above, or the read chamber may be in fluidic communication with said compartment.

The microfluidics device may further include one or more additional compartments containing other solutions, such as (but not limited to) wash solutions, calibration solutions, quality control solutions, dilution solutions, excipients, interference solutions, positive controls, negative controls, and the like. These additional compartment(s) may be in fluidic communication with one or more of the other compartments. For example, the microfluidics device may further include one or more compartments containing a wash solution, and these compartment(s) may be capable of being in fluidic communication with any other compartment(s) of the device. In another example, the microfluidics device may further include one or more compartments containing an excipient for dissolution of one or more dried reagents, and the compartment(s) may be capable of being in fluidic communication with any other compartment(s) of the device. In yet a further example, the microfluidics device may include one or more compartments containing a dilution solution, and the compartment(s) may be capable of being in fluidic communication with any other compartment(s) of the device.

Certain non-limiting embodiments of the present disclosure are directed to a method of detecting the presence and/or concentration of antibodies to a microorganism in a human biological sample. In one non-limiting embodiment, the method comprises the steps of: (1) combining, either simultaneously or wholly or partially sequentially, (a) the human biological sample suspected of containing antibodies to the microorganism, (b) a composition comprising a singlet oxygen-activatable chemiluminescent compound having at least one antigen of the microorganism directly or indirectly bound thereto, and (c) a composition comprising a sensitizer capable of generating singlet oxygen in its excited state and at least one anti-human immunoglobulin antibody directly or indirectly bound to the sensitizer; (2) allowing the binding of (b) and (c) to antibodies to the microorganism present in (a), wherein the binding of (b) and (c) to the antibodies to the microorganism results in the formation of a complex in which the sensitizer is brought into close proximity to the chemiluminescent compound; (3) activating the sensitizer to generate singlet oxygen, wherein activation of the sensitizers present in the complex causes the activation of the chemiluminescent compound present in each complex; and (4) determining the amount of chemiluminescence generated by the activated chemiluminescent compound in the complex to determine the presence and/or concentration of antibodies to the microorganism present in the sample.

In another non-limiting embodiment, the method comprises the steps of: (1) combining, either simultaneously or wholly or partially sequentially: (a) the human biological sample suspected of containing antibodies to the microorganism; (b) a composition comprising a singlet oxygen-activatable chemiluminescent compound having at least one antigen of the microorganism directly or indirectly bound thereto; (c) a composition comprising a sensitizer capable of generating singlet oxygen in its excited state and a biotin-specific binding partner directly or indirectly bound thereto; and (d) at least one anti-human immunoglobulin antibody, wherein the antibody is biotinylated; (2) allowing the binding of (c) to (d) to form (c)/(d) and the binding of (b) and (c)/(d) to antibodies to the microorganism present in (a), wherein the binding of (b) and (c)/(d) to the antibodies to the microorganism results in the formation of a complex in which the sensitizer is brought into close proximity to the chemiluminescent compound; (3) activating the sensitizer to generate singlet oxygen, wherein activation of the sensitizers present in the complex causes the activation of the chemiluminescent compound present in each complex; and (4) determining the amount of chemiluminescence generated by the activated chemiluminescent compound in the complex to determine the presence and/or concentration of antibodies to the microorganism present in the sample.

Other non-limiting embodiments of the method comprises the steps of: (1) combining, either simultaneously or wholly or partially sequentially, (a) the human biological sample suspected of containing antibodies to the microorganism, (b) a composition comprising a singlet oxygen-activatable chemiluminescent compound having at least one anti-human immunoglobulin antibody directly or indirectly bound thereto, and (c) a composition comprising a sensitizer capable of generating singlet oxygen in its excited state and at least one antigen of the microorganism directly or indirectly bound to the sensitizer; (2) allowing the binding of (b) and (c) to antibodies to the microorganism present in (a), wherein the binding of (b) and (c) to the antibodies to the microorganism results in the formation of a complex in which the sensitizer is brought into close proximity to the chemiluminescent compound; (3) activating the sensitizer to generate singlet oxygen, wherein activation of the sensitizers present in the complex causes the activation of the chemiluminescent compound present in each complex; and (4) determining the amount of chemiluminescence generated by the activated chemiluminescent compound in the complex to determine the presence and/or concentration of antibodies to the microorganism present in the sample.

In yet another non-limiting embodiment, the method comprises the steps of: (1) combining, either simultaneously or wholly or partially sequentially: (a) the human biological sample suspected of containing antibodies to the microorganism; (b) a composition comprising a singlet oxygen-activatable chemiluminescent compound having at least one anti-human immunoglobulin antibody directly or indirectly bound thereto; (c) a composition comprising a sensitizer capable of generating singlet oxygen in its excited state and a biotin-specific binding partner directly or indirectly bound thereto; and (d) at least one antigen of the microorganism, wherein the antigen is biotinylated; (2) allowing the binding of (c) to (d) to form (c)/(d) and the binding of (b) and (c)/(d) to antibodies to the microorganism present in (a), wherein the binding of (b) and (c)/(d) to the antibodies to the microorganism results in the formation of a complex in which the sensitizer is brought into close proximity to the chemiluminescent compound; (3) activating the sensitizer to generate singlet oxygen, wherein activation of the sensitizers present in the complex causes the activation of the chemiluminescent compound present in each complex; and (4) determining the amount of chemiluminescence generated by the activated chemiluminescent compound in the complex to determine the presence and/or concentration of antibodies to the microorganism present in the sample.

Any anti-microorganism antibody capable of detection via the assay formats described or otherwise contemplated herein may be detected by the serology assays of the present disclosure. In a particular (but non-limiting) embodiment, the antibody is an anti-viral antigen antibody for the SARS-COV-2 virus.

In some non-limiting assay embodiments, signal producing system (sps) members are utilized that comprise a sensitizer such as, for example, a photosensitizer, and a chemiluminescent molecule composition, and each of which have either the antigen of the microorganism or the anti-human immunoglobulin antibody directly or indirectly attached thereto (or are capable of having the antigen or antibody directly or indirectly attached thereto during the assay); in these assay embodiments, activation of the sensitizer results in a product that activates the chemiluminescent composition(s), thereby generating a detectable signal that relates to the amount of bound human anti-microorganism antibody being detected. An exemplary (but non-limiting) embodiment of an assay platform on which the present disclosure can be based is the Luminescence Oxygen Channeling Assay (LOCI®; Siemens Healthcare Diagnostics Inc., Tarrytown, NY). The LOCI® assay is described, for example, in U.S. Pat. No. 5,340,716 (Ullman et al.), the entire contents of which are expressly incorporated herein by reference.

Any of the microorganism antigens, anti-human immunoglobulin antibodies, singlet oxygen-activatable chemiluminescent compounds, sensitizers, fluorescent molecules, and biotin or analogs thereof described in detail herein above or otherwise contemplated herein may be utilized in the methods of the present disclosure.

For example, in certain particular (but non-limiting) embodiments, the singlet oxygen-activatable chemiluminescent compound is a substance that undergoes a chemical reaction with singlet oxygen to form a metastabile intermediate species that can decompose with the simultaneous or subsequent emission of light.

In particular (but non-limiting) embodiments, the sensitizer is a photosensitizer, and the activation of the sensitizer in step (3) comprises irradiation with light (such as, but not limited to, irradiation at about 680 nm).

Any sample for which an assay for the presence of antibodies to a microorganism is desired can be utilized as the sample in accordance with the methods of the present disclosure. Non-limiting examples of samples include a biological sample such as, but not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof. Particular non-limiting examples include lysed whole blood cells and lysed red blood cells. In particular (but non-limiting) examples, the sample is from a human.

As mentioned above, the various components of the method are provided in combination (either simultaneously or sequentially). When the various components of the method are added sequentially, the order of addition of the components may be varied; a person having ordinary skill in the art can determine the particular desired order of addition of the different components to the assay. The simplest order of addition, of course, is to add all the materials simultaneously and determine the signals produced therefrom. Alternatively, each of the components, or groups of components, can be combined sequentially. In certain embodiments, one or more steps (such as, but not limited to, one or more incubation step(s) and/or one or more wash step(s)) may be involved subsequent to one or more additions.

In an alternative (but non-limiting) exemplary embodiment, step (1) of the method that utilizes a chemiluminescent detection system includes first combining the sample with the biotinylated anti-human immunoglobulin antibody and the composition comprising the sensitizer and incubating same before adding the composition comprising the singlet oxygen-activatable chemiluminescent compound and microbial antigen. Alternatively, step (1) of the method can include first combining the sample with the composition comprising the singlet oxygen-activatable chemiluminescent compound and incubating same before adding the composition comprising the sensitizer. In this latter embodiment, the biotinylated anti-human immunoglobulin antibody may be added before or after the incubation step. Note that these different orders of addition are also applicable when the antigen is biotinylated and the anti-human immunoglobulin antibody is associated with the chemiluminescent compound.

While the above embodiments have been described with respect to detecting antibodies in human samples, it will be understood that scope of the present disclosure is not limited to use with human samples. Rather, any of the embodiments described herein above can be adapted for detection of antibodies to a microorganism in biological samples from other mammals. Thus, the present disclosure also includes compositions, kits, devices, and methods for detection of anti-microbial antibodies in non-human mammalian biological samples by substituting the anti-human immunoglobulin antibodies utilized in all of the embodiments described above with anti-mammalian immunoglobulin antibodies that correspond to the species from which the biological sample is taken.

### Example

An Example is provided hereinbelow. However, the present disclosure is to be understood to not be limited in its application to the specific experimentation, results, and laboratory procedures disclosed herein. Rather, the Example is simply provided as one of various embodiments and are meant to be exemplary, not exhaustive.

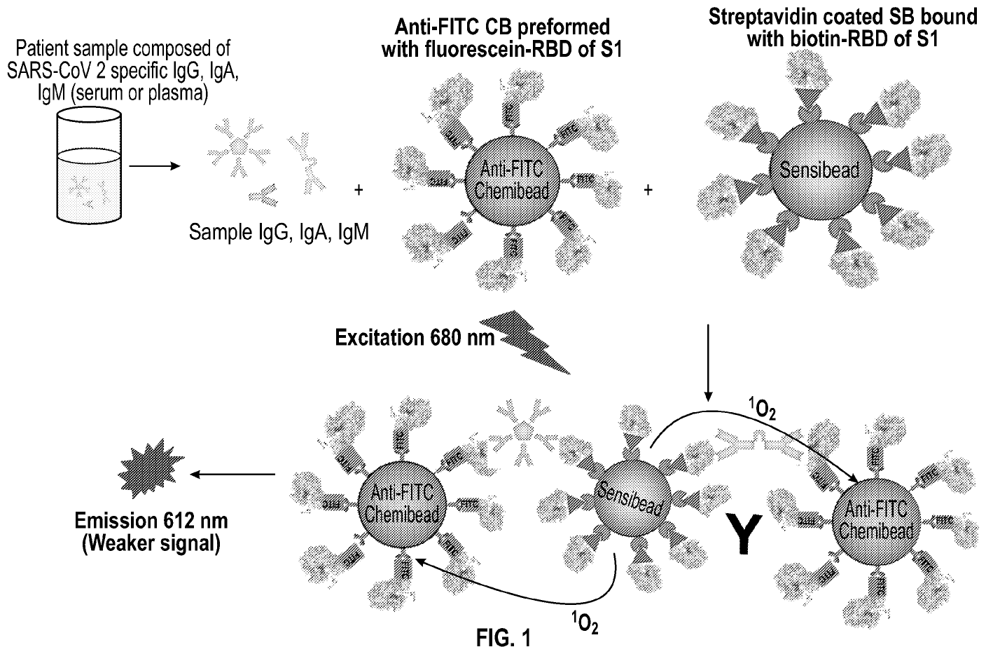

In June 2020, the U.S. Food and Drug Administration (FDA) issued an Emergency Use Authorization (EUA) for a laboratory-based total antibody test developed by Siemens Healthineers (Malvern, PA) for the detection of the presence of SARS-COV-2 antibodies, including IgM, IgA, and IgG, in blood. A spike protein on the surface of the SARS-COV-2 virus enables the virus to penetrate and infect human cells found in multiple organs and blood vessels. The Siemens Healthineers' Total Antibody COV2T assay is shown in FIG. 1 and was designed to detect antibodies to the spike protein. Some of these antibodies are believed to neutralize the SARS-COV-2 virus and therefore prevent infection. Multiple potential vaccines in development for SARS-COV-2 include the spike protein within their focus.

The assay format of FIG. 1 utilizes two reagents that both contain SARS-COV-2 antigens: a first reagent comprising anti-FITC chemibeads preformed with the fluorescein-labeled receptor binding domain of the S1 subunit of SARS-COV-2 spike protein (RBD of S1), and a second reagent comprising streptavidin-coated sensibeads bound with biotin-RBD of S1. Note that the biotinylated-RBD of S1 antigen can be produced as a separate reagent from the universal streptavidin-coated sensibeads and combined to produce the second assay reagent at any point prior to or during the assay.

However, the bridging of two viral antigen molecules (i.e., the two RBD of S1 antigens depicted in FIG. 1) by a monomer bivalent antibody (such as IgG) present in the biological sample may produce a weak signal due to slower Brownian movement and the uncertainty of the anti-viral antibody orientation upon attachment to a first antigen. Further, given the presence of multiple antigen molecules on each chemibead, it is possible that both binding arms of the patient's anti-RBD antibody could bind to the surface of a single chemibead. As a result, there is a need to enhance the detection signal of the bridging assay, especially for samples that have a low anti-viral antibody titer, as well as provide a mechanism for detection of anti-SARS-COV-2 antibodies where both binding arms thereof are bound to the surface of a single chemibead.

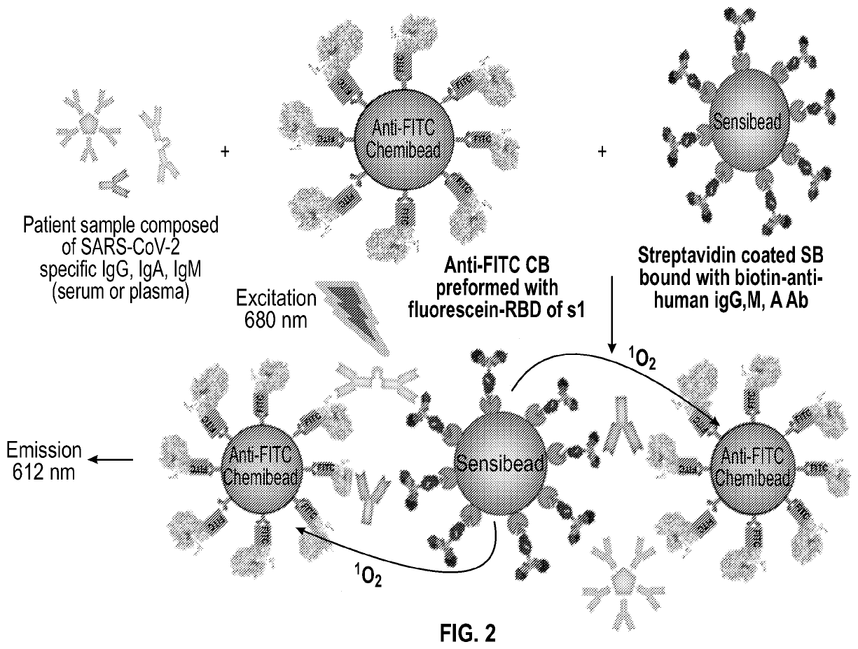

FIG. 2 illustrates one embodiment of an assay format constructed in accordance with the present disclosure that addresses these issues. In this assay format, a biotinylated anti-human immunoglobulin antibody was added in the reaction mixture in lieu of the biotinylated viral antigen (biotin-RBD of S1) of FIG. 1 for the measurement of total anti-RBD antibodies in patient samples. In this Example, the antibody utilized was a commercially available affinity purified goat polyclonal antibody against human IgG/M/A obtained from Thermo Fisher Scientific (Rockford, IL; Catalog number A18849).

This method provides another way of measuring the total anti-RBD antibodies (including anti-RBD IgG, IgM, and IgA) versus the bridging assay format (i.e., RBD-coated chemibead (CB) and RBD-coated sensibead (SB)) of FIG. 1. The advantage of this approach is that it overcomes steric hindrance issues with anti-RBD antibody binding to two antigen molecules located on two different beads and also eliminates the potential issue of anti-RBD antibody failing to bridge chemibead and sensibead when both binding arms of the patient's anti-RBD antibody could bind to the chemibead surface.

Table 1 contains data demonstrating the detection of anti-SARS-COV-2 antibody in COVID-19 positive samples using the assay format of the present disclosure (as illustrated in FIG. 2, and as labeled “Biotin-anti-human AMG” in Table 1), and compared to detection of anti-SARS-COV-2 antibody in COVID-19 positive samples using the assay format of FIG. 1 (labeled “Biotin-RBD of S1” in Table 1).

TABLE 1

| | | |
|---|---|---|
| | Biotin-RBD of S1 | Biotin-anti-human AMG |
| Samples Tested | Index >1000 is positive | Index >1000 is positive |

COVID-19 Positive Samples

| | | |
|---|---|---|
| B7 | 1124 | 2139 |
| B8 | 2407 | 2427 |
| B10 | 1051 | 1578 |
| B13 | 235 | 1086 |
| B15 | 398 | 1314 |
| B16 | 843 | 1684 |
| B19 | 1600 | 2934 |
| B20 | 1410 | 3093 |
| B21 | 1449 | 3237 |
| B22 | 761 | 1560 |
| B23 | 16484 | 9962 |
| S10338 | 35655 | 15184 |
| S9 | 444 | 1346 |
| S17 | 545 | 2104 |
| S22 | 1355 | 2565 |

COVID-19 Negative Samples

| | | |
|---|---|---|
| P19 | 742 | 891 |
| N46 | 32 | 580 |
| N47 | 29 | 765 |
| N48 | 30 | 740 |
| N49 | 31 | 744 |
| N50 | 28 | 748 |
| N51 | 29 | 756 |
| API Positive | 569 | 1607 |
| Survey Sample | | |
| MP2-CV2T | | 1082 |

Note that the term “anti-human AMG” used in Table 1 refers to an anti-human antibody that specifically binds to all of IgA, IgM, and IgG, and thus provides for detection of total anti-SARS-COV2 antibodies in COVID-19 patient samples (where “total” refers to the sum of IgG, IgM, and IgA).

As can be seen in Table 1, a positive result for anti-SARS-COV-2 antibodies was detected in nine (9) out of the 15 COVID-19 positive samples tested using the assay format of FIG. 1. In contrast, a positive result for anti-SARS-COV-2 antibodies was detected in all 15 of the COVID-19 positive samples tested using the assay format of the present disclosure (as depicted in FIG. 2). In addition, both assay formats provided a negative result for all seven (7) COVID-19 negative samples tested.

Therefore, the present disclosure provides compositions, kits, microfluidics devices, and methods that are effective in detecting antibodies to microorganisms in biological samples. In particular (but not by way of limitation), this Example demonstrates that the compositions, kits, microfluidics devices, and methods of the present disclosure are highly effective in detecting anti-SARS-COV-2 antibodies in biological samples.

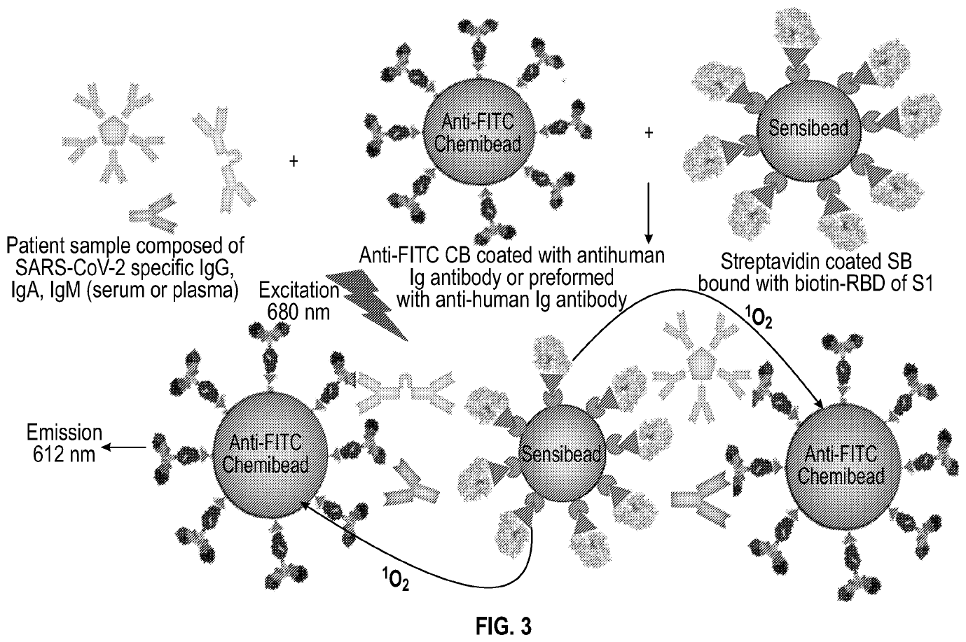

As discussed in detail herein above, it should be noted that the attachment of the antigen and anti-human immunoglobulin antibody to the other two components (i.e., chemibead and sensibead) in FIG. 2 is arbitrary; indeed, the present disclosure also includes the opposite of the attachments described herein above. For example, FIG. 3 illustrates another non-limiting embodiment of an assay format constructed in accordance with the present disclosure. The assay format of FIG. 3 is identical to the assay format of FIG. 2, with the exception that the two compounds bound to the chemibead and sensibead are switched. That is, the assay format of FIG. 3 utilizes a chemibead having at least one anti-human immunoglobulin antibody directly or indirectly bound thereto and a sensibead having an antigen of the microorganism directly or indirectly bound to the sensitizer (wherein the sensibead may have a biotin-specific binding partner directly or indirectly bound thereto, and the antigen is biotinylated for subsequent attachment to the sensibead).

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A kit for performing a serology assay that detects the presence and/or concentration of antibodies to a microorganism in a human biological sample utilizing a chemiluminescent detection system, the kit comprising:
(a) a composition comprising a singlet oxygen-activatable chemiluminescent compound having an antigen of the microorganism directly or indirectly bound thereto; and
(b) a composition comprising a sensitizer capable of generating singlet oxygen in its excited state and at least one anti-human immunoglobulin antibody directly or indirectly bound to the sensitizer.

2. The kit of claim 1, wherein the microorganism is a virus.

3. The kit of claim 2, wherein the virus is severe acute respiratory syndrome coronavirus 2 (SARS-COV-2).

4. The kit of claim 1, wherein the at least one anti-human immunoglobulin antibody specifically binds to human IgA, human IgG, and human IgM.

5. A kit for performing a serology assay that detects the presence and/or concentration of antibodies to a microorganism in a human biological sample utilizing a chemiluminescent detection system, the kit comprising:
(a) a composition comprising a singlet oxygen-activatable chemiluminescent compound having an antigen of the microorganism directly or indirectly bound thereto;
(b) a composition comprising a sensitizer capable of generating singlet oxygen in its excited state and a biotin-specific binding partner directly or indirectly bound thereto; and
(c) at least one anti-human immunoglobulin antibody, wherein the antibody is biotinylated.

6. The kit of claim 5, wherein the microorganism is a virus.

7. The kit of claim 6, wherein the virus is severe acute respiratory syndrome coronavirus 2 (SARS-COV-2).

8. The kit of claim 5, wherein the at least one anti-human immunoglobulin antibody specifically binds to human IgA, human IgG, and human IgM.

9. A microfluidics device for detecting the presence and/or concentration of antibodies to a microorganism in a human biological sample, the microfluidics device comprising:
(i) an inlet channel through which the human biological sample is applied;
(ii) at least a first compartment capable of being in fluidic communication with the inlet channel and containing:
(a) a composition comprising a singlet oxygen-activatable chemiluminescent compound having an antigen of the microorganism directly or indirectly bound thereto; and
(b) at least one composition comprising a sensitizer capable of generating singlet oxygen in its excited state, and at least one anti-human immunoglobulin antibody capable of being directly or indirectly bound to the sensitizer.

10. The microfluidics device of claim 9, wherein the microorganism is a virus.

11. The microfluidics device of claim 10, wherein the virus is severe acute respiratory syndrome coronavirus 2 (SARS-COV-2).

12. The microfluidics device of claim 9, wherein the at least one anti-human immunoglobulin antibody of (b) specifically binds to human IgA, human IgG, and human IgM.

13. The microfluidics device of claim 9, wherein (b) is further defined as:
a composition comprising a sensitizer capable of generating singlet oxygen in its excited state and a biotin-specific binding partner directly or indirectly bound thereto; and
at least one anti-human immunoglobulin antibody, wherein the antibody is biotinylated.

14. The microfluidics device of claim 9, wherein (a) and (b) are present in the same compartment.

15. The microfluidics device of claim 9, wherein (a) and (b) are in two different compartments.

16. A method for detecting the presence and/or concentration of antibodies to a microorganism in a human biological sample, the method comprising the steps of:
(1) combining, either simultaneously or wholly or partially sequentially:
(a) the human biological sample suspected of containing antibodies to the microorganism;
(b) a composition comprising a singlet oxygen-activatable chemiluminescent compound having at least one antigen of the microorganism directly or indirectly bound thereto; and
(c) a composition comprising a sensitizer capable of generating singlet oxygen in its excited state and at least one anti-human immunoglobulin antibody directly or indirectly bound to the sensitizer;
(2) allowing the binding of (b) and (c) to antibodies to the microorganism present in (a), wherein the binding of (b) and (c) to the antibodies to the microorganism results in the formation of a complex in which the sensitizer is brought into close proximity to the chemiluminescent compound;
(3) activating the sensitizer to generate singlet oxygen, wherein activation of the sensitizers present in the complex causes the activation of the chemiluminescent compound present in each complex; and
(4) determining the amount of chemiluminescence generated by the activated chemiluminescent compound in the complex to determine the presence and/or concentration of antibodies to the microorganism present in the sample.

17. The method of claim 16, wherein the microorganism is a virus.

18. The method of claim 17, wherein the virus is severe acute respiratory syndrome coronavirus 2 (SARS-COV-2).

19. The method of claim 16, wherein the at least one anti-human immunoglobulin antibody of (c) specifically binds to human IgA, human IgG, and human IgM.

20. The method of claim 16, wherein the human biological sample is selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.

21. A method for detecting the presence and/or concentration of antibodies to a microorganism in a human biological sample, the method comprising the steps of:
(1) combining, either simultaneously or wholly or partially sequentially:
(a) the human biological sample suspected of containing antibodies to the microorganism;
(b) a composition comprising a singlet oxygen-activatable chemiluminescent compound having at least one antigen of the microorganism directly or indirectly bound thereto;
(c) a composition comprising a sensitizer capable of generating singlet oxygen in its excited state and a biotin-specific binding partner directly or indirectly bound thereto; and
(d) at least one anti-human immunoglobulin antibody, wherein the antibody is biotinylated;
(2) allowing the binding of (c) to (d) to form (c)/(d) and the binding of (b) and (c)/(d) to antibodies to the microorganism present in (a), wherein the binding of (b) and (c)/(d) to the antibodies to the microorganism results in the formation of a complex in which the sensitizer is brought into close proximity to the chemiluminescent compound;
(3) activating the sensitizer to generate singlet oxygen, wherein activation of the sensitizers present in the complex causes the activation of the chemiluminescent compound present in each complex; and
(4) determining the amount of chemiluminescence generated by the activated chemiluminescent compound in the complex to determine the presence and/or concentration of antibodies to the microorganism present in the sample.

22. The method of claim 21, wherein the microorganism is a virus.

23. The method of claim 22, wherein the virus is severe acute respiratory syndrome coronavirus 2 (SARS-COV-2).

24. The method of claim 21, wherein the at least one anti-human immunoglobulin antibody of (c) specifically binds to human IgA, human IgG, and human IgM.

25. The method of claim 21, wherein the human biological sample is selected from the group consisting of whole blood or any portion thereof, urine, saliva, sputum, cerebrospinal fluid, skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof.

26.-50. (canceled)
